(19) United States
(12) Patent Application Publication
(10) Pub. No.: US 2014/0295407 A1
(43) Pub. Date: Oct. 2, 2014

(54) BIOSENSOR BASED ON THE INNATE IMMUNE SYSTEM

(71) Applicant: Los Alamos National Security, LLC, Los Alamos, NM (US)

(72) Inventors: Basil I. Swanson, Los Alamos, NM (US); Harshini Mukundan, Los Alamos, NM (US); Anu Chaudhary, Seattle, WA (US)

(73) Assignee: Los Alamos National Security, LLC

(21) Appl. No.: 14/228,170

(22) Filed: Mar. 27, 2014

Related U.S. Application Data
(60) Provisional application No. 61/806,071, filed on Mar. 28, 2013.

(51) Int. Cl. G01N 33/569

(52) U.S. Cl. CPC G01N 33/56911; G01N 33/56983; G01N 33/56938
USPC 435/5; 435/7.37; 435/288.7; 435/7.33

(57) ABSTRACT

Described herein are biosensors for early, pre-symptomatic detection of infectious agents and methods for their use. In particular, this disclosure describes biosensors that utilize toll-like receptor (TLR) binding domains to detect pathogen-associated molecular patterns (PAMPs). Also provided herein are methods of detecting and/or capturing a PAMP from a biological sample using the disclosed biosensors.

### CROSS-REFERENCE TO RELATED APPLICATIONS

This application claims the benefit of U.S. Provisional Application No. 61/806,071, filed Mar. 28, 2013, which is herein incorporated by reference in its entirety.

### ACKNOWLEDGMENT OF GOVERNMENT SUPPORT

This invention was made with government support under Contract No. DE-AC52-06NA25396 awarded by the U.S. Department of Energy. The government has certain rights in the invention.

### FIELD

This disclosure concerns biosensors for early, pre-symptomatic detection of infectious microorganisms. In particular, this disclosure concerns biosensors that utilize toll-like receptor (TLR) binding domains to detect pathogen-associated molecular patterns (PAMPs).

### BACKGROUND

Three types of methods are currently available for detecting infections—monitoring the toxic effects on host cells caused by the infectious agent (culture); fingerprinting the DNA of the infectious agent; and detecting signature molecules of an infectious agent or of the patient's late immune response (antibodies). While some of these methods can identify the infectious agent (DNA fingerprinting), all of them have a long turn-around time; either the assay takes too long (i.e. culture), or the assay is dependent on detecting markers that only develop measurable concentrations late in the infection. All of these techniques generally require critical laboratory equipment and measurement at off-site labs.

Innate immunity is the first-line host defense of multicellular organisms that rapidly operates to limit infection upon exposure to infectious agents. The cells and molecules operating during this early stage of the immune response have a decisive impact on shaping the subsequent adaptive immune response that takes several days to develop. The initial recognition of microorganisms is mediated by a set of germ-line encoded receptors, the toll-like receptors (TLRs). Thirteen mammalian TLRs have been identified thus far. These innate immune receptors exhibit specificities for conserved molecular structures, pathogen-associated molecular patterns (PAMPs), which are shared by classes of pathogens. While PAMPs are diverse in their structure (the category includes CpG DNA, double stranded RNA, protein, lipoprotein, and lipopolysaccharides, for instance), they have several characteristic features important for their immunogenic activity. They are produced by microorganisms, but not by the host, and are essential for the survival of the microorganism. PAMPs often represent a molecular signature of a pathogen class. Thus, recognition of PAMPs by the innate immune system not only signals the presence of infection, but also provides valuable information regarding the class of infecting pathogen.

Members of the TLR family are expressed differentially among host cells and have been shown to respond to different PAMPs (Barton and Medzhitov, Curr Top Microbiol Immunol 270:81-92, 2002). While TLR9 binds unmethylated CpG DNA from bacteria, TLR3 and TLR5 are activated by viral double stranded RNA and bacterial flagellin, respectively. TLR4 responds to lipopolysaccharide (LPS) of gram-negative bacteria, whereas TLR2 is promiscuous and interacts with bacterial lipoproteins (lipoteichoic acid (LTA), peptidoglycan, lipoarabinomannans, lipopeptide, as well as atypical LPS). The increasing number of molecules recognized by TLR2 is most likely due to the cooperation of TLR2 with TLR1 or TLR6. In all cases, multiple TLRs aggregate upon binding PAMPs (Ozinsky et al., Proc Natl Acad Sci USA 97: 13766-13771, 2000). In the simplest case, either the same TLR aggregates (homodimer) or two different TLRs aggregate (heterodimer) upon binding a PAMP molecule.

### SUMMARY

Provided herein is a method of detecting the presence of at least one pathogen-associated molecular pattern (PAMP) molecule or putative PAMP molecule in a biological sample. In some embodiments, the method includes providing a lipid assembly, wherein the lipid assembly comprises one or more toll-like receptors (TLRs) partitioned into or associated with the lipid assembly; exposing the lipid assembly to the biological sample for an amount of time sufficient for the at least one PAMP molecule or putative PAMP molecule to bind the TLR(s); and detecting binding of the PAMP or putative PAMP to the TLRs. Further provided is a method of diagnosing a subject with a microbial infection by detecting the presence of at least one PAMP molecule in a biological sample from the subject according to the methods disclosed herein. The presence of the PAMP molecule in the biological sample diagnoses the subject as having a microbial infection.

Also provided is a method of capturing at least one PAMP molecule or putative PAMP molecule from a biological sample. In some embodiments, the method includes providing a lipid assembly, wherein the lipid assembly comprises one or more TLRs partitioned into or associated with the lipid assembly; exposing the lipid assembly to the sample for an amount of time sufficient for the at least one PAMP molecule or putative PAMP molecule to bind the TLRs; harvesting the lipid assembly with the one or more bound PAMP molecules or putative PAMP molecules; and separating the one or more PAMP molecules or putative PAMP molecules from the lipid assembly.

Further provided is a method of assessing disease state in a first subject by providing a biological sample from the first subject; analyzing the biological sample according to the method disclosed herein to produce a test PAMP profile for the sample; comparing the test PAMP profile for the sample with a second PAMP profile and drawing a conclusion about the disease state of the first subject based on differences or similarities between the test PAMP profile and the second PAMP profile. In some embodiments, the second PAMP profile is for a second sample taken from the first subject at a different time point, or a sample taken from a second subject.

Also provided herein is a lipid assembly comprising one or more TLRs partitioned into or associated with the lipid assembly. In some embodiments, the lipid assembly comprises a substantially planar lipid structure (for example, a supported lipid bilayer (SLB), a tethered bilayer lipid membrane (t-BLM), a self-assembled monolayer (SAM), or a combination thereof), a vesicle (such as a multilamellar vesicle, a unilamellar vesicle, or a mixture thereof), a liposome, a nanodisc, a bicelle, or a micelle. In some examples, the substantially planar lipid structure is upon a functionalized waveguide surface.

Further provided is a biosensor comprising a lipid assembly on a functionalized waveguide surface, wherein the lipid assembly comprises one or more TLRs partitioned into or associated with the lipid assembly. In some embodiments, the lipid assembly comprises a substantially planar lipid structure (for example, a SLB, a t-BLM, a SAM, or a combination thereof), a vesicle (such as a multilamellar vesicle, a unilamellar vesicle, or a mixture thereof), a liposome, a nanodisc, a bicelle, or a micelle.

The foregoing and other objects, features, and advantages of the invention will become more apparent from the following detailed description, which proceeds with reference to the accompanying figures.

### BRIEF DESCRIPTION OF THE DRAWINGS

FIG. 1 is a schematic of the membrane-based FRET assay. An excitation laser propagates down a planar optical waveguide. The evanescent tail of the guided light penetrates into the bilayer membrane exciting the yellow reporter dye. Upon binding, the red (square) and the yellow (circle) dye are brought into close proximity. The green dye loses its excitation energy via fluorescence resonant energy transfer to the red dye. The red fluorescence increases while the yellow dye fluorescence decreases.

FIG. 2 is a schematic illustrating representative ways in which PAMPs can be used to track disease progression. Expression of pathogen-specific PAMPs changes during the course of a disease, and can be used to predict conversion (latent to active), response to treatment, emergence of drug resistance, relapse of infection, and so forth. Though the figure is illustrated with reference to early secretory antigen 6 (ESAT-6) and LAM, it is believed that other PAMPs (including in other diseases) will also vary such that the levels and set of PAMPs detected in a subject sample at any time (or over a course of time) can be used to track disease characteristics in that subject.

FIG. 3 shows a schematic of early detection of PAMPs using waveguide based TLR array. From left to right: TLR2 heterodimerizes with TLR1, TLR4, or TLR6 to recognize products from gram-positive bacteria; TLR3 heterodimerizes with an unknown TLR upon binding double stranded RNA (dsRNA) from viruses; TLR4 homodimerizes in binding LPS from gram-negative bacteria and requires surface molecules such as LBP and CD14 for recognition; and TLR5 homodimerizes to recognize bacterial flagellin.

FIG. 4 is a schematic of a generic membrane anchor.

FIG. 5 is a graph showing partitioning of TLR4 into a lipid assembly on a waveguide surface. Purified extracellular TLR4 (eTLR4) was flooded through the waveguide with a supported DOPC bilayer containing 0.1% biotin. Fluorescently-labeled eTLR4 antibody was added. Measurement of fluorescence intensity indicated that eTLR4 was inserted into the lipid bilayer surface.

### DETAILED DESCRIPTION

### I. Abbreviations

CARD caspase recruitment domain

CFP-10 culture filtrate protein 10

DOPC 1,2-dioleoyl-sn-glycero-3-phosphocholine

dsRNA double-stranded RNA

ESAT-6 early secretory antigen 6

eTLR extracellular domain of TLR

FCS fluorescence correlation spectroscopy

FRET fluorescence resonance energy transfer

FTID flow-through ion deposition

HDL high density lipoprotein

LAM lipoarabinomannans

LDL low density lipoprotein

LPS lipopolysaccharide

LRR leucine-rich repeat

NOD nucleotide-binding oligomerization domain

PAMP pathogen-associated molecular pattern

PGL-I phenolic glycolipid 1

PRR pattern recognition receptor

SAM self-assembled monolayer

SLB supported lipid bilayer

t-BLM tethered bilayer lipid membrane

TLR toll-like receptor

### II. Terms and Methods

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Affinity molecule or affinity ligand: A ligand/molecule that binds a selected target molecule/moiety specifically and reversibly. Antibodies are one example of an affinity molecule, which selectively bind the antigen to which they were raised. The biotin/streptavidin pair is another example.

Amphipathic: An amphipathic molecule contains both lipophilic/hydrophobic (non-polar) and lipophobic/hydrophilic (polar) groups/moieties. Such a compound is called amphiphilic or an amphiphile. The lipophilic portion of an amphipathic compound is able to insert at least partially into a lipid structure, such as a lipid bilayer, monolayer, micelle, or vesicle.

Without intending to be bound to any particular structure, the hydrophobic group in an amphiphile may be an alkyl group, such as a long carbon chain, for example, with the formula: CH₃(CH₂)ₙ, (where n is generally greater than or equal to about 4 to about 16). Such carbon chains also optionally comprise one or more branches, wherein one hydrogen is replaced with an aliphatic moiety, such as an alkyl group. A hydrophobic group also can comprise an aryl group. The hydrophilic group/portion of an amphiphile comprises one or more of the following: a peptide or protein, a carbohydrate, an ionic molecule, such as an anionic molecule (e.g., a fatty acid, a sulfate or a sulfonate) or a cationic molecule, an amphoteric molecule (e.g., a phospholipid), or a non-ionic molecule (e.g., a small polymer). One of ordinary skill in the art will understand that the term amphiphile encompasses myriad different combinations of hydrophilic (water soluble) and hydrophobic (lipid soluble) moieties.

Antibody: A polypeptide ligand comprising at least a light chain or heavy chain immunoglobulin variable region which specifically recognizes and binds an epitope (e.g., an antigen). This includes intact immunoglobulins and the variants and portions of them well known in the art, such as Fab′ fragments, F(ab)′₂ fragments, single chain Fv proteins (“scFv”), disulfide stabilized Fv proteins (“dsFv”), diabodies (dimers of scFv fragments), and minibodies (fusions of scFv and CH3 domain). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (e.g., humanized murine antibodies), and heteroconjugate antibodies (e.g., bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

Typically, an immunoglobulin has a heavy and light chain. Each heavy and light chain contains a constant region and a variable region (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs has been defined according to Kabat et al. (see, Kabat et al., Sequences of Proteins of Immunological Interest, U.S. Department of Health and Human Services, 1991) and the ImMunoGeneTics database (IMGT) (see, Lefranc, Nucleic Acids Res 29:207-9, 2001). The Kabat and IMGT databases are maintained online. The sequences of the framework regions of different light or heavy chains are relatively conserved within a species. The framework region of an antibody, that is the combined framework regions of the constituent light and heavy chains, serves to position and align the CDRs in three-dimensional space.

CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

References to “V_{H}” or “VH” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor,” and the human immunoglobulin providing the framework is termed an “acceptor.” In one embodiment, all of the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Humanized immunoglobulins can be constructed by means of genetic engineering (e.g., see U.S. Pat. No. 5,585,089).

Biological sample: Any biological material, such as a fluid produced from or obtained from an organism, a cell, a collection of cells (e.g., cultured cells), a tissue sample, a biopsy, or an organism. Biological samples also include blood and blood products (e.g., plasma) and other biological fluids (e.g., tears, sweat, sputum, saliva and related fluids, urine, tears, mucous, and so forth). Tissue samples can be from any organ or tissue in the body, including heart, liver, muscle, adipose, brain, lung, testes, and brain.

Biological samples may be from individual subjects (e.g., animals, such as humans, mice, rats, monkeys, marmosets, chickens, cats, dogs, pigs, guinea pigs, horses, cows, fruit flies, or worms) and/or archival repositories. The samples may be acquired directly from the individuals, from clinicians (for instance, who have acquired the sample from the individual), or directly from archival repositories.

Biomarker: A substance (or set of substances) used as an indicator of a biological state, most commonly a disease. In many instances, biomarkers are biomolecules that are differentially expressed during the course of disease. In the case of infectious disease, example biomarkers are pathogen-associated biomolecules, such as PAMPs, that are secreted in the host during infection. Many biomarkers are virulence factors required for pathogenicity of the infectious agent and several are expressed very early in disease onset.

Cardiolipin (CL): (IUPAC name: 1,3-bis(sn-3′-phosphatidyl)-sn-glycerol) An important component of the inner mitochondrial membrane, where it constitutes about 20% of the total lipid composition. The name ‘cardiolipin’ is derived from the fact that it was first identified in animal hearts. Cardiolipin is essential for the optimal function of numerous enzymes that are involved in mitochondrial energy metabolism.

Culture filtrate protein 10 (CFP-10): The protein encoded by the esxB gene. CFP-10 is a 10 kDa antigen secreted from Mycobacterium tuberculosis. It forms a 1:1 heterodimeric complex with ESAT-6. Both genes are expressed from the RD1 region of the bacterial genome and play a key role in virulence of the infection. CFP-10 is also known as ESAT-6-like protein esxB or secreted antigenic protein MTSA-10.

Early secretory antigen 6 (ESAT-6): ESAT-6 is a 6 kDa early secretory antigenic target of Mycobacterium tuberculosis. ESAT-6 forms a 1:1 heterodimeric complex with CFP-10. It is a potent T cell antigen, and is used in tuberculosis diagnosis by the whole blood interferon γ test QuantiFERON®-TB Gold (QFT), in conjunction with CFP-10 and TB7.7.

Flagellin: The basic element of bacterial flagella, surface structures on bacteria (such as gram negative bacteria) that are involved in motility. Flagellin has a molecular weight of approximately 40,000 Daltons, and is composed of subunits arranged in several-stranded helix formation somewhat resembling myosin in structure. Exemplary flagellin proteins are described, for example, in U.S. Pat. Nos. 6,585,980; 6,130,082; 5,888,810; 5,618,533; and 4,886,748; U.S. Patent Publication No. US 2003/0044429; and Donnelly et al., J. Biol. Chem. 43: 40456, 2002, all incorporated herein by reference. Natural flagellin includes (i) a flagellin N-terminal constant region; (ii) a flagellin C-terminal constant region; and (iii) a flagellin hypervariable region between the two constant regions.

Hydrophobic: A hydrophobic (or lipophilic) group is electrically neutral and nonpolar, and thus prefers other neutral and nonpolar solvents or molecular environments. Examples of hydrophobic molecules include alkanes, oils and fats.

Hydrophilic: A hydrophilic (or lipophobic) group is electrically polarized and capable of H-bonding, enabling it to dissolve more readily in water than in oil or other “non-polar” solvents.

Infectious disease: Any disease caused by an infectious agent. Examples of infectious agents (also referred to herein as “infectious pathogens” or “pathogens”) include, but are not limited to: viruses, bacteria, mycoplasma and fungi. In a particular example, it is a disease caused by at least one type of infectious agent (or infectious pathogen). In another example, it is a disease caused by at least two different types of infectious pathogens. Infectious diseases can affect any body system, be acute (short-acting) or chronic/persistent (long-acting), occur with or without fever, strike any age group, and overlap each other. Infectious diseases can be opportunistic infections, in that they occur more frequently in immunocompromised subjects

Examples of infectious bacteria include: Helicobacter pylori, Borelia burgdorferi, Legionella sps including Legionella pneumophilia, Mycobacteria sps (such as. M. tuberculosis, M. avium, M. bovis, M. intracellulare, M. kansaii, M. gordonae, M. leprae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, corynebacterium diphtheriae, corynebacterium sp., Erysipelothrix rhusiopathiae, Escherichia coli, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Actinomyces israelli, Vibrio cholerae, Yersinia pestis, Mycobacterium leprae, Salmonella typhimurium, Campylobacter jejuni, Helicobacter pylori, Haemophilus influenza, and Pseudomonas sps. Also contemplated are gram negative bacteria having lipopolysaccharide and any gram positive bacteria having lipoteichoic acid.

Label: A detectable compound or composition that is conjugated directly or indirectly to another molecule (such as a nucleic acid molecule or protein, for instance an antibody) to facilitate detection of that molecule. Examples of labels include, but are not limited to, radioactive isotopes, enzyme substrates, co-factors, ligands, chemiluminescent agents, fluorophores, haptens, enzymes, and combinations thereof. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998).

Lipid: As used herein, the term lipid refers to a class of water-insoluble, oily or greasy organic substances that are extractable from cells and tissues by nonpolar solvents, such as chloroform or ether. The most abundant kinds of lipids are fats or triacylglycerols, which are major fuels for most organisms. Another class of lipids is the polar lipids, which are major components of cell membranes. The following table (Table 1) provides one way (by chemical structure) of grouping major types of lipids:

TABLE 1

| Lipid type | Representative examples or sub-groups |
|---|---|
| Triacylglycerols | |
| Waxes | |
| Phosphoglycerides | phosphatidylethanolamine |
| | phosphatidylcholine |
| | phosphatidylserine |
| | phosphatidylinositol |
| | cardiolipin |
| Sphingolipids | sphingomyelin |
| | cerebrosides |
| | gangliosides |
| Sterols and their | (see Table 3) |
| fatty acid esters | |

Lipids may also be broken down into other recognized classes, such as those shown in Table 2.

TABLE 2

| SCIENTIFIC NAME | ABBREVIATION |
|---|---|
| Lyso-Phosphatidylcholine | LY |
| Sphingomyelin | SP |
| Phosphatidylcholine | PC |
| Phosphatidylserine | PS |
| Phosphatidylinositol | PI |
| Phosphatidylethanolamine | PE |
| Cardiolipin | CL |
| Free Fatty Acids | FFA |
| Monoacylglycerides | MAG |
| Diacylglycerides | DAG |
| Triacylglycerides | TAG |
| Cholesterol Esters | CE |
| Phosphatidic acids | PA |
| Phosphatidylglycerols | PG |
| CDP-diacylglycerols | CDP-DAG |
| Lysocardiolipin | LyCL |
| Lysophosphatidylethanolamine | LyPE |

Also included in the term lipid are the compounds collectively known as sterols. Table 3 shows representative sterols.

TABLE 3

| | MOLECULAR | |
|---|---|---|
| SCIENTIFIC NAME | FORMULA | COMMON NAME |
| 5b-cholestan-3b-ol | C₂₇H₄₈O | coprostanol |
| 5a-cholestan-3b-ol | C₂₇H₄₈O | dihydrocholesterol |
| 5-cholesten-3b-ol | C₂₇H₄₆O | cholesterol |
| 5,24-cholestadien-3b-ol | C₂₇H₄₄O | desmosterol |
| 5-cholestan-25a-methyl-3b-ol | C₂₈H₄₂O | campesterol |
| 5-cholestan-24b-methyl-3b-ol | C₂₈H₄₂O | dihydrobrassicasterol |
| 5-cholesten-24b-ethyl-3b-ol | C₂₉H₅₀O | b-sitosterol |
| 5,22-cholestadien-24b-ethyl- | C₂₉H₄₈O | stigmasterol |
| 3b-ol | | |

Lipid A: A lipid component of an endotoxin responsible for toxicity of Gram-negative bacteria. It is the innermost of the three regions of a lipopolysaccharide (LPS, also called endotoxin) molecule; its hydrophobic nature allows it to anchor the LPS to the outer membrane (Raetz & Whitfield, Annu Rev. Biochem 71(1)635-700, 2002). While its toxic effects can be damaging, the sensing of lipid A by the immune system may also be important for the onset of immune responses to Gram-negative infection, and for the subsequent successful fight against the infection.

Lipid assembly: A broad term that encompasses all structures that include lipid molecules, including particularly mono-layers and bi-layers, substantially planar structures, vesicles (unilamellar or multilamellar, liposomes, micelles, nanodiscs, and bicelles, for instance). Also included in this term are supported lipid bilayers (SLB), tethered bilayer lipid membranes (t-BLM), and self-assembled monolayers (SAM), as well as naturally occurring or synthetic HDL particles, naturally occurring or synthetic LDL particles, or a mixture of any two or more of any of these.

The term “lipid assembly” encompasses naturally occurring lipid structures (e.g., HDL or LDL particles extracted from blood, cell membranes, and so forth), as well as synthetic lipid constructs, both planar and vesicular and otherwise, whether made from purified lipidic compounds or defined or undefined mixtures of lipidic compounds.

Lipoarabinomannan (LAM): A lipoglycan (a lipid to which a carbohydrate is attached) and major virulence factor in the bacterial genus Mycobacterium, including M. tuberculosis.

Lipopeptide: A molecule comprising both a peptide moiety and at least one lipid (acyl) moiety. Many microbial species contain in their inner and outer membranes and/or cell walls amphipathic lipids based on one or two amino acids linked to one (monoacyl), two (diacyl) or three (triacyl) fatty acids. Diacyl lipopeptides and triacyl lipopeptides are known PAMPs recognized by TLRs.

Lipophilic: The term lipophilic refers to the ability of a chemical compound to insert into (partition into) a lipid structure such as a lipid bi-layer; a lipophilic compound can dissolve in fats, oils, lipids, and non-polar solvents such as hexane or toluene. Lipophilic substances interact within themselves and with other substances through the London dispersion force. They have little to no capacity to form hydrogen bonds. When a molecule of a lipophilic substance is enveloped by water, surrounding water molecules enter into an ice-like structure over the greater part of its molecular surface; this thermodynamically unfavorable event drives oily substances out of water. Thus lipophilic substances tend to be water insoluble.

Lipopolysaccharide (LPS): Also known as lipoglycans, lipopolysaccharides are large molecules consisting of a lipid and a polysaccharide joined by a covalent bond; they are found in the outer membrane of Gram-negative bacteria, act as endotoxins and elicit strong immune responses in animals. LPS comprises three parts: the O antigen (or O polysaccharide; a repetitive glycan polymer), the core oligosaccharide, and lipid A. The exact structure of LPS in a bacterial cell wall can be species or strain specific.

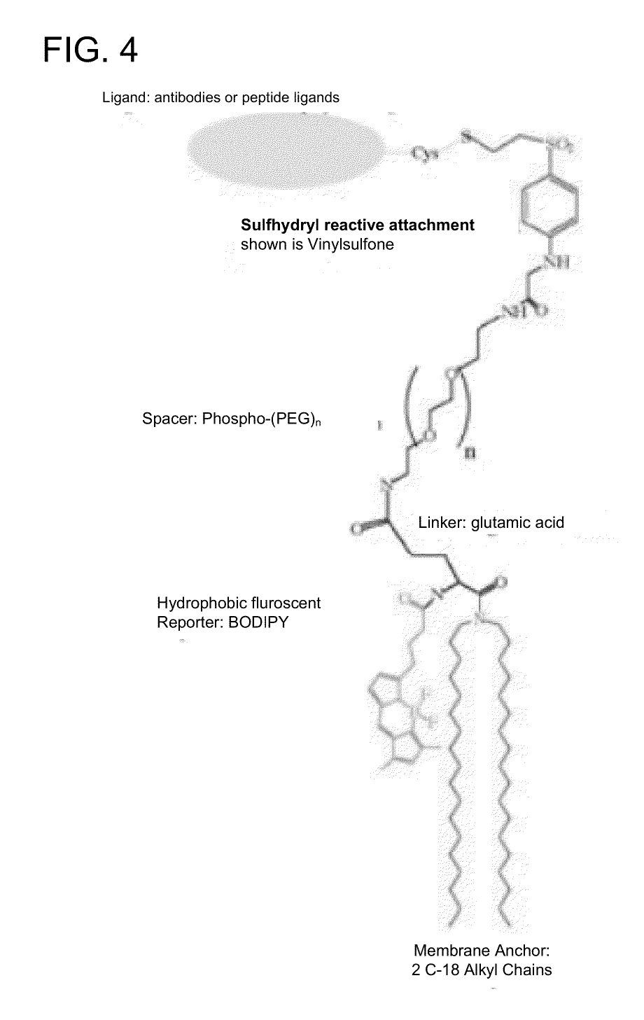

Membrane anchor: A hydrophobic molecule capable of insertion into a lipid bilayer or lipid assembly. An exemplary membrane anchor is depicted in FIG. 4. In some embodiments, a TLR or eTLR is conjugated to a membrane anchor for insertion into a lipid assembly.

Microbe: Any type of microorganisms, including bacteria, viruses, fungi and mycoplasma. A “microbial infection” is an infection caused by a microbe.

Mycobactin T: (IUPAC name: [4-[(1-hydroxy-2-oxoazepan-3-yl)amino]-4-oxobutan-2-yl]6-[henicosanoyl(hydroxy)amino]-2-[[(2E)-2-(6-oxocyclohexa-2,4-dien-1-ylidene)-1,3-oxazolidine-4-carbonyl]amino]hexanoate) An iron binding compound produced by bacteria of the genus Mycobacterium. See, e.g., Snow, Bacteriol Rev. 34(2):99-125, 1970.

Pathogen-associated molecular pattern (PAMP): Biomarkers that are recognized by the early innate immune system in response to infection. Bacterial PAMPs are amphiphiles that possess a common structural motif that facilitates partitioning into phospholipid bilayers. These molecules can be referred to as small molecular motifs conserved within a class of microbes. They are recognized by toll-like receptors (TLRs) and other pattern recognition receptors (PRRs) in both plants and animals, and stimulate (activate) a TLR response in cell-based assays.

PAMPs activate innate immune responses, protecting the host from infection, by identifying some conserved non-self molecules. Bacterial lipopolysaccharide (LPS), an endotoxin found on the bacterial cell membrane of a bacterium, is considered to be the prototypical PAMP. LPS is specifically recognized by TLR4, a recognition receptor of the innate immune system. Other PAMPs include bacterial flagellin (recognized by TLR5), lipoteichoic acid from Gram-positive bacteria, peptidoglycan, and nucleic acid variants normally associated with viruses, such as double-stranded RNA (dsRNA), recognized by TLR3 or unmethylated CpG motifs, recognized by TLR9.

The term “PAMP” is somewhat of a misnomer, as most microbes, not only pathogens, express the molecules detected; the term microbe-associated molecular pattern (Ausubel, Nature Immun. 6(1):973-979, 2005)), or MAMP (Didielaurent et al., Cell Mol. Life. Sci. 62(2):1285-1287, 2006), has therefore been proposed. A virulence signal capable of binding to a pathogen receptor, in combination with a MAMP, has been proposed as one way to constitute a (pathogen-specific) PAMP (Rumbo et al., FEBS Letters 580(12):2976-2984, 2006). Plant immunology frequently treats the terms PAMP and MAMP interchangeably, considering them to be the first step in plant immunity, PTI (PAMP-triggered immunity) (Jones & Dangl, Nature 444(7117):323-329, 2006).

In various embodiments, a PAMP molecule is selected from the group consisting of flagellin, lipid A, cardiolipin, di-acyl lipopeptide, tri-acyl lipopeptide, peptidoglycan, lipoarabinomannan (LAM), phenolic glycolipid 1 (PGL-I), mycobactin T, lipopolysaccharide (LPS) and culture filtrate protein 10 (CFP-10).

PAMP fingerprint or profile: A distinct or identifiable pattern of PAMP levels, for instance a pattern of high and low level PAMPs in a defined set, such as a stage of a (bacterial) disease, presence or absence of (bacterial) infection, and so forth. PAMP profiles or fingerprints (also referred to as linked profiles, e.g., a disease-linked profile or disease stage-linked profile) can be linked to particular bacterial infection, to a particular stage of bacterial disease development (or infection by at least one bacterium along with co-infection by at least one other organism), normal (non-infected) subject samples (including subjects “infested” with one or more non-pathogenic bacterial species), antibiotic susceptibility or resistance, or to any other distinct or identifiable condition that influences production/release and/or levels of PAMP molecules (e.g., concentrations) in a predictable or associatable way.

PAMP profiles/fingerprints can include relative as well as absolute levels of specific PAMP molecules. The set of PAMP molecules and levels thereof in an individual sample is referred to as the individual PAMP profile of that sample, which serves as a molecular signature not unlike a genomic profile or metabolomics profile—though a PAMP profile is specific for an infection or state of infection and so forth.

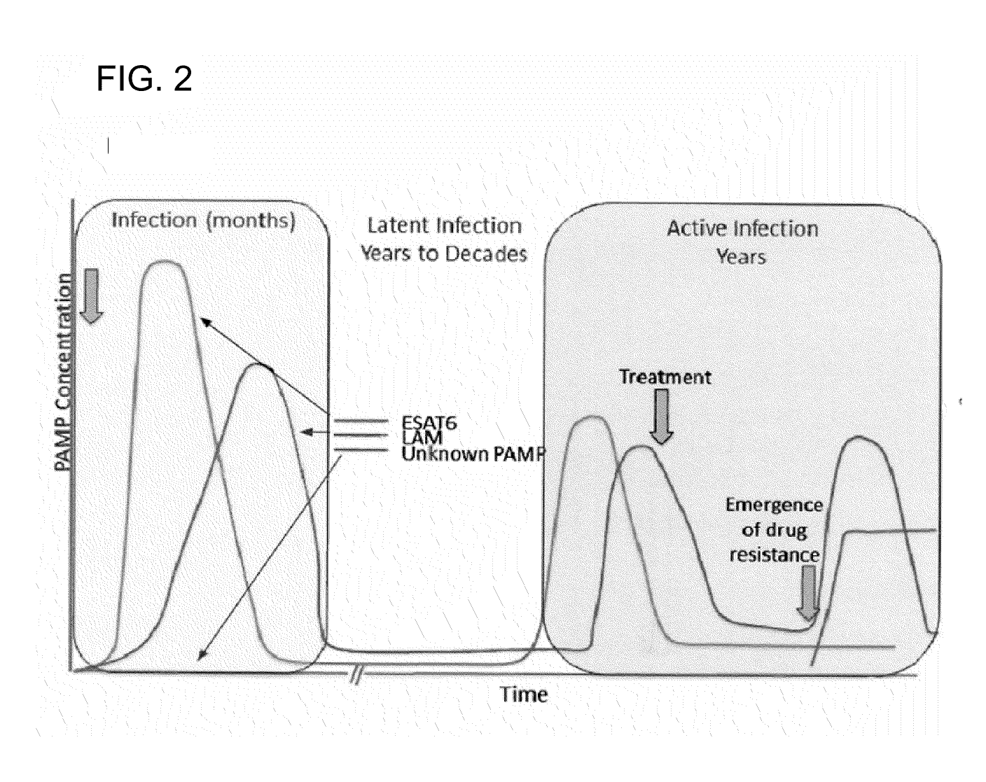

It is also contemplated that a “profile” may refer to the longitudinal change in PAMP molecule levels through time. FIG. 2, for instance, illustrates a longitudinal profile of PAMP levels as they change through time.

Peptidoglycan: Also known as murein, peptidoglycan is a polymer consisting of sugars and amino acids that forms a mesh-like layer outside the plasma membrane of bacteria, forming the cell wall. The sugar component consists of alternating residues of β-(1,4) linked N-acetylglucosamine and N-acetylmuramic acid. Attached to the N-acetylmuramic acid is a peptide chain of three to five amino acids; the peptide can be cross-linked to the peptide chain of another strand forming the 3D mesh-like (cross-linked) layer. Peptidoglycan serves a structural role in the bacterial cell wall, giving strength as well as counteracting the osmotic pressure of the cytoplasm.

Phenolic glycolipid: A class of mycoside compound produced by Mycobacterium and comprising an oligosaccharide moiety linked to a phenolphthiocerol molecule mainly esterified by mycoserosic acids. Phenolic glycolipids are immunogenic with their carbohydrate at the non-reducing end. PGL-I is a major antigen characteristic of M. leprae, forming a loose extracellular capsule around the bacillus. PGL-I is a suspected PAMP.

Pattern recognition receptors (PRR): A class of innate immune response-expressed proteins that respond to pathogen-associated molecular patterns (PAMP) and endogenous stress signals termed danger-associated molecular patterns (DAMP). Pattern recognition receptors (PRRs) include, for example, membrane-associated PRR (such as TLRs, which sense pathogen-associated or danger-associated molecular patterns extracellularly or in endosomes and receptors may link innate and adaptive immune responses); cytoplasmic PRRs of the CATERPILLER family (also known as NACHT-leucine-rich repeat (NLR) proteins) (e.g., Nucleotide-binding oligomerization domain proteins (NODs) recognize intracellular MDP (muramyl dipeptide) and transduce signals via NF-κB and MAP kinase pathways through the serine/threonine kinase RIP2. The nucleotide-binding oligomerization domain binds nucleotide triphosphate. NODs signal via N-terminal caspase recruitment (CARD) domains to activate downstream gene induction events; pyrin domain-containing proteins (NALPs) contain a nucleotide binding site (NBS) for nucleotide triphosphates plus C-terminal leucine-rich repeats (LRRs), which appear to act as a regulatory domain and may be involved in the recognition of microbial pathogens. NALPs appear to recognize endogenous or microbial molecules or stress responses and to form oligomers with caspase-1, which cleave IL-1 into its active form; RNA helicases-LGP2 acts as a dominant-negative inhibitor, and RIG-I and Mda5 activate antiviral signaling. These RNA Helicases recruit factors via twin N-terminal CARD domains, activate antiviral gene programs; and plant R proteins that share structural and functional similarity with PRRs found in higher animals); and secreted PRRs (such as complement receptors, collectins; pentraxin proteins (including serum amyloid P component (SAP), acute-phase C-reactive protein (CRP), cytokine-modulated PTX3); lipid transferases; and peptidoglycan recognition proteins (PGRs), which are critical for insect immunity, and but less well characterized in mammals).

Synthetic: Prepared or made artificially. In the context of the present disclosure, a “synthetic” lipid assembly is a lipid assembly that is not naturally occurring.

Toll-like receptors (TLRs): Type I transmembrane proteins characterized by an extracellular domain containing leucine-rich repeats (LRRs) and a cytoplasmic tail that contains a conserved region called the Toll/IL-1 receptor (TIR) domain, which protein acts as a pattern recognition receptor (PRR). Toll-like receptors play a role in innate immunity, for example, by recognizing conserved microbial structures or PAMPs. Thirteen TLRs (named TLR1 to TLR13) have been identified thus far. However, equivalents of certain TLRs found in humans are not present in all mammals. For example, a gene coding for a protein analogous to TLR10 in humans is present in mice, but appears to have been modified by a retrovirus. On the other hand, mice express TLRs 11, 12, and 13, none of which are represented in humans.

Representative nucleic acid sequences that encode human TLRs, and corresponding protein sequences are publically available, e.g., as shown in Table 4 (all GENBANK™ numbers referred to herein are incorporated by reference for the sequence as it was publicly available on Aug. 28, 2012). Naturally occurring and artificial ligands of several TLRs have been characterized. Exemplary ligands are listed in Table 4; see also “Toll-Like Receptors (TLRs) and Innate Immunity” in Handbook of Experimental Pharmacology, 183:1-20, 2008. A TLR ligand is said to “activate” a TLR receptor or “stimulate” TLR pathway activity if the ligand binds to the receptor, and such binding results in the initiation of one or more signaling events, such as translocation or phosphorylation of the TLR receptor and/or other signaling molecules.

TABLE 4
Exemplary TLR sequences and ligands

| | GenBank nucleic | GenBank | | | |
|---|---|---|---|---|---|
| | acid sequences | protein sequences | Ligands | Cell types | Location |
| TLR1 | U88540; | AAC34137; | multiple triacyl | monocytes/macrophages; | cell surface |
| | AB445617.1; | AB445617; | lipopeptides | a subset of dendritic | |
| | BC141321.1 | AAI41320.1 | | cells; B lymphocytes | |
| TLR2 | U88878; | AAC34133.1; | multiple | monocytes/macrophages; | cell surface |
| | NM_011905.3 | AAD49335.1 | glycolipids, | myeloid dendritic cells; | |
| | | | lipopeptides, and | mast cells | |
| | | | lipoproteins; | | |
| | | | lipoteichoic acid; | | |
| | | | HSP70; zymosan | | |
| | | | (beta-glucan); | | |
| | | | MALP-2; HSP70 | | |
| TLR3 | U88879; | AAC34134.1; | poly I: C; | dendritic cells; B | cell |
| | NG_007278.1; | BAG55028.1; | poly(I: C₁₂U); | lymphocytes | compartment |
| | NM_126166.4 | AAH99937.1 | dsRNA (a viral | | |
| | | | product) | | |
| TLR4 | U88880; | AAC34135.1; | lipopolysaccharides | monocytes/macrophages; | cell surface |
| | NG_011475 | CAH72619.1; | (LPS); | myeloid dendritic cells; | |
| | | CAH72618.1; | peptidoglycan | mast cells; intestinal | |
| | | AAD29272.1 | fragments | epithelium | |
| | | | (glycopeptides); | | |
| | | | several heat shock | | |
| | | | proteins; | | |
| | | | fibrinogen; heparan | | |
| | | | sulfate fragments; | | |
| | | | hyaluronic acid | | |
| | | | fragments | | |
| TLR5 | AB060695.1; | ACM69034.1; | flagellin | monocyte/macrophages; | cell surface |
| | BC125247 | BAB43955.1; | | a subset of dendritic | |
| | | AAI25248.1; | | cells; intestinal | |
| | | NP_058624.2 | | epithelium | |
| TLR6 | AB020807; | ABY67133.1; | multiple diacyl | monocytes/macrophages; | cell surface |
| | EU195556.1; | NP_035734.3 | lipopeptides | mast cells; B | |
| | NM_011604.3 | | | lymphocytes | |
| TLR7 | AF245702; | AAF78035.1; | gardiquimod; | monocytes/macrophages; | cell |
| | AK313858 | BAG36586.1; | single stranded | plasmacytoid dendritic | compartment |
| | | CAM14953.1 | RNA (such as viral | cells; B lymphocytes | |
| | | | RNA); | | |
| | | | bropirimine; | | |
| | | | loxoribine; | | |
| | | | imidazoquinoline; | | |
| | | | imiquimod; | | |
| | | | resiquimod | | |
| TLR8 | AF245703; | AAF78036.1; | single stranded | monocytes/macrophages; | cell |
| | BC132054.1 | CAM14949.1 | RNA (such as viral | a subset of dendritic | compartment |
| | | | RNA); resiquimod | cells; mast cells | |
| TLR9 | AB045181.1; | AAF78037.1; | CpG | monocytes/macrophages; | cell |
| | AF245704; | BAB19260.1; | oligonucleotides; | plasmacytoid dendritic | compartment |
| | AF259262; | AAK28488.1 | unmethylated CpG | cells; B lymphocytes | |
| | AF259263 | | DNA (such as | | |
| | | | those found in the | | |
| | | | genome of bacteria | | |
| | | | and viruses) | | |
| TLR10 | AF296673; | AAK26744.1; | | monocytes/macrophages; | cell surface |
| | AB445680.1; | BAG55077.1; | | B lymphocytes | |
| | NM_001146035.1 | NP_001139507 | | | |
| TLR11 | FJ539013.1; | AAS37672.1; | Profilin | monocytes/macrophages; | cell |
| | AY510704.1 | ACL80330.1 | | liver cells; kidney; | compartment |
| | | | | bladder epithelium | |
| TLR12 | NM_001108682.1; | NP_001102152.1; | | | |
| | NM_205823.2 | AAS37673.1 | | | |
| TLR13 | NM_205820.1 | AAS37674.1 | | | cell |
| | | | | | compartment |

TLRs that are activated by bacterial PAMPs (e.g., TLR1, TLR2, TLR3, TLR4, TLR5, TLR6 and TLR9) are more relevant to the assays described herein, and particularly to the systems described for identifying and characterizing new PAMPs.

TLRs play a critical role in the early innate immune response to invading pathogens by sensing microorganisms. These evolutionarily conserved receptors, homologues of the Drosophila Toll gene, recognize highly conserved structural motifs only expressed by microbial pathogens, called PAMPs. PAMPs include various bacterial cell wall components such as lipopolysaccharide (LPS), peptidoglycan (PGN) and lipopeptides, as well as flagellin, bacterial DNA and viral double-stranded RNA.

Stimulation of TLRs by PAMPs initiates signaling cascades that involve a number of proteins, such as MyD88, TRIF and IRAK (Medzhitov et al., Nature, 388(6640):394-7, 1997). These signaling cascades lead to the activation of transcription factors, such as AP-1, NF-κB and IRFs, inducing the secretion of pro-inflammatory cytokines and effector cytokines that direct the adaptive immune response.

TLRs are predominantly expressed in tissues involved in immune function, such as spleen and peripheral blood leukocytes, as well as those exposed to the external environment, such as lung and the gastrointestinal tract. Their expression profiles vary among tissues and cell types. TLRs are located on the plasma membrane with the exception of TLR3, TLR7 and TLR9 which are localized intracellularly (Nishiya & DeFranco et al., J Biol Chem. 279(18):19008-17, 2004).

Ten human and twelve murine TLRs have been characterized, TLR1 to TLR10 in humans, and TLR1 to TLR9, TLR11, TLR12 and TLR13 in mice, the homolog of TLR10 being a pseudogene. TLR2 is essential for the recognition of a variety of PAMPs from Gram-positive bacteria, including bacterial lipoproteins, lipomannans and lipoteichoic acids. TLR3 is implicated in virus-derived double-stranded RNA. TLR4 is predominantly activated by lipopolysaccharide. TLR5 detects bacterial flagellin. TLR9 is required for response to unmethylated CpG DNA. TLR7 and TLR8 recognize small synthetic antiviral molecules (Jurk et al., Nat Immunol, 3(6):499, 2002), and recently single-stranded RNA was reported to be their natural ligand (Heil et al., Science. 303(5663):1526-9, 2004). TLR11(12) has been reported to recognize uropathogenic E. coli (Zhang et al., Science. 303:1522-1526, 2004) and a profilin-like protein from Toxoplasma gondii (Lauw et al., Trends Immunol. 26(10):509-11, 2005).

The repertoire of specificities of the TLRs is apparently extended by the ability of TLRs to heterodimerize with one another. For example, dimers of TLR2 and TLR6 are required for responses to diacylated lipoproteins while TLR2 and TLR1 interact to recognize triacylated lipoproteins (Ozinsky et al., PNAS USA, 97(25):13766-71, 2000). Specificities of the TLRs are also influenced by various adapter and accessory molecules, such as MD-2 and CD14 that form a complex with TLR4 in response to LPS (Miyake et al., Int Immunopharmacol. 3(1):119-28, 2003).

TLRs are single transmembrane proteins containing an extracellular domain, a short membrane spanning region and a cytosolic (intracellular) portion. The presence of PAMPs is detected by the extracellular domain, which aggregates upon binding. In some embodiments of the present disclosure, the extracellular domain of a TLR—referred to as an “eTLR”—is used for insertion into a lipid assembly, such as by conjugation to a membrane anchor which is inserted into the lipid assembly.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

Applicants hereby incorporate by reference the entirety of the following co-owned applications into this application: U.S. application Ser. No. 13/529,847, filed Jun. 21, 2012; U.S. Provisional Application No. 61/499,665 filed on Jun. 21, 2011; U.S. application Ser. No. 12/658,298, filed Feb. 8, 2010; U.S. Provisional Application No. 61/206,980, filed Feb. 6, 2009; and U.S. Provisional Application No. 61/251,605, filed Oct. 14, 2009.

### III. Overview of Several Embodiments

Provided herein is a method of detecting the presence of at least one pathogen-associated molecular pattern (PAMP) molecule or putative PAMP molecule in a biological sample. In some embodiments, the method includes providing a lipid assembly, wherein the lipid assembly comprises one or more toll-like receptors (TLRs) partitioned into or associated with the lipid assembly; exposing the lipid assembly to the biological sample for an amount of time sufficient for the at least one PAMP molecule or putative PAMP molecule to bind the TLRs; and detecting binding of the PAMP or putative PAMP to the TLRs, thereby detecting the presence of the PAMP or putative PAMP in the biological sample.

Further provided is a method of diagnosing a subject with a microbial infection by detecting the presence of at least one PAMP molecule in a biological sample from the subject according to the methods disclosed herein. The presence of the PAMP molecule in the biological sample diagnoses the subject with a microbial infection. In some embodiments, the microbial infection is a bacterial infection, such as an infection by Gram-positive bacteria, Gram-negative bacteria and/or motile bacteria (for example, bacteria with flagella). In other embodiments, the microbial infection is a viral infection.

In particular examples in which the microbial infection is an infection with Gram-positive bacteria, for example, Staphylococcus aureus, the lipid assembly comprises TLR2 and TLR1, TLR4 or TLR6. When TLR2 heterodimerizes with TLR1, TLR4 or TLR6, the complex is capable of recognizing peptidoglycan from Gram-positive bacteria.

In specific examples in which the microbial infection is an infection with Gram-negative bacteria, such as E. coli, the lipid assembly comprises TLR4. When TLR4 homodimerizes, the complex is capable of recognizing LPS from Gram-negative bacteria.

In particular examples in which the microbial infection is an infection with motile bacteria, the lipid assembly comprises TLR5. When TLR5 homodimerizes, the complex is capable of recognizing bacterial flagellin.

In specific examples in which the microbial infection is an infection with a virus, the lipid assembly comprises TLR3 and another TLR. When TLR3 heterodimerizes with the other TLR, the complex is capable of recognizing dsRNA from viruses.

Also provided herein is a method of capturing at least one PAMP molecule or putative PAMP molecule from a biological sample. In some embodiments, the method includes providing a lipid assembly, wherein the lipid assembly comprises one or more TLRs partitioned into or associated with the lipid assembly; exposing the lipid assembly to the sample for a sufficient time for the at least one PAMP molecule or putative PAMP molecule to bind the TLRs; harvesting the lipid assembly with the one or more bound PAMP molecules or putative PAMP molecules; and separating the one or more PAMP molecules or putative PAMP molecules from the lipid assembly.

Further provided is a method of assessing disease state in a first subject by providing a biological sample from the first subject; analyzing the biological sample according to the method disclosed herein to produce a test PAMP profile for the sample; comparing the test PAMP profile for the sample with a second PAMP profile and drawing a conclusion about the disease state of the first subject based on differences or similarities between the test PAMP profile and the second PAMP profile. In some embodiments, the second PAMP profile is for a second sample taken from the first subject at a different time point, or a sample taken from a second subject.

In particular embodiments of the disclosed methods, the lipid assembly comprises a substantially planar lipid structure, a vesicle, a liposome, a nanodisc, a bicelle, or a micelle. In some examples, the substantially planar lipid structure comprises a supported lipid bilayer (SLB), a tethered bilayer lipid membrane (t-BLM), a self-assembled monolayer (SAM), or a combination thereof. In one non-limiting example, the substantially planar lipid structure is upon a functionalized waveguide surface. In some examples, the vesicle is a multilamellar vesicle, a unilamellar vesicle, or a mixture thereof.

In some embodiments of the disclosed methods, the PAMP molecule is selected from the group consisting of cardiolipin, culture filtrate protein 10 (CFP-10), di-acyl lipopeptide, flagellin, lipoteichoic acid, lipid A, lipoarabinomannan (LAM), lipomannan, lipopolysaccharide (LPS), mycobactin T, peptidoglycan, phenolic glycolipid I (PGL-I), and tri-acyl lipopeptide.

In other embodiments, the putative PAMP molecule is characterized by stimulating TLR pathway activity in a cell-based TLR activity assay, being present in a sample from a subject exposed to or infected by a bacterial pathogen, or both.

In some embodiments of the methods, the biological sample is from a subject suspected of being infected with an infectious agent.

In some embodiments, the biological sample is a biological fluid sample, for example a blood sample or blood product sample. In other embodiments, the biological sample is a cell or tissue sample, such as from a biopsy.

In some embodiments of the disclosed methods, the one or more TLRs are selected from TLR1, TLR2, TLR3, TLR4, TLR5, TLR6, TLR7, TLR8, TLR9, TLR10, TLR11, TLR12 and TRL13. In particular embodiments, the one or more TLRs are selected from TLR1, TLR2, TLR3, TLR4, TLR5, TLR6 and TLR9. In particular examples, the one or more TLRs comprise TLR4.

In some embodiments, the one or more TLRs are recombinant TLRs comprising or consisting of the extracellular domain of the TLRs (eTLRs). In some examples, the one or more TLRs or eTLRs are conjugated to a membrane anchor which is inserted into the lipid assembly.

Also provided herein is a lipid assembly comprising one or more TLRs partitioned into or associated with the lipid assembly. In some embodiments, the lipid assembly comprises a substantially planar lipid structure (for example, a supported lipid bilayer (SLB), a tethered bilayer lipid membrane (t-BLM), a self-assembled monolayer (SAM), or a combination thereof), a vesicle (such as a multilamellar vesicle, a unilamellar vesicle, or a mixture thereof), a liposome, a nanodisc, a bicelle, or a micelle. In some examples, the substantially planar lipid structure is upon a functionalized waveguide surface.

Further provided is a biosensor comprising a lipid assembly on a functionalized waveguide surface, wherein the lipid assembly comprises one or more TLRs partitioned into or associated with the lipid assembly. In some embodiments, the lipid assembly comprises a substantially planar lipid structure (for example, a SLB, a t-BLM, a SAM, or a combination thereof), a vesicle (such as a multilamellar vesicle, a unilamellar vesicle, or a mixture thereof), a liposome, a nanodisc, a bicelle, or a micelle.

### IV. Lipid Assembly and TLR-Based Sensor Assays

A compact, integrated sensor for the detection of biological agents based upon materials that mimic cell membranes and their receptors has been developed (Song et al., J Am Chem Soc 120:11514-11515, 1998; Song and Swanson, Langmuir 15:4710-4712, 1999; Song and Swanson, Anal Chem 285:35-41, 2000; Song and Swanson, Anal Chem 71:2097-2107, 1999).

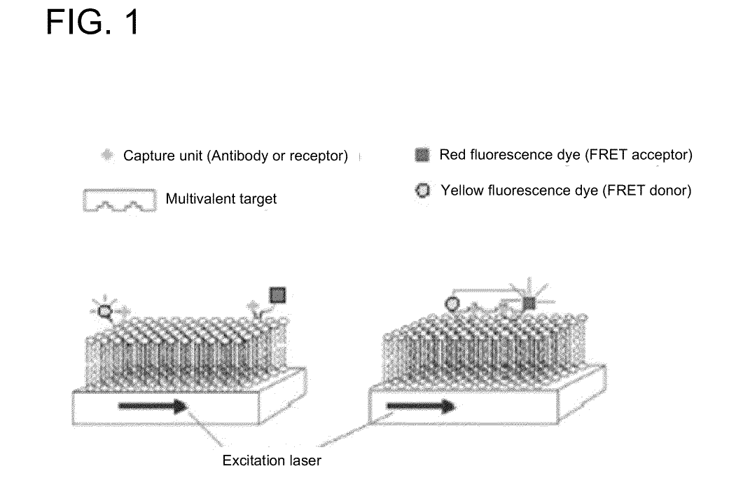

The biosensor approach is depicted schematically in FIG. 1. This approach uses a lipid bilayer decorated with fluorescently labeled recognition molecules for the target of interest. In earlier work, the recognition molecules were either the natural receptors of the pathogen or affinity ligands selected against the target of interest. In the FRET assay (FIG. 1), lipid bilayers containing membrane-anchored, fluorescently labeled receptors are directly deposited on the top of a planar optical waveguide. An evanescent wave from the guided light is used to excite the donor molecule of the FRET pair. The fluorescence of both the donor and acceptor labeled receptors are measured. Target binding is signaled by a decrease in fluorescence intensity of the donor and an increase in intensity of the acceptor. The present disclosure describes the use of the same membrane-based FRET approach where the recognition molecules (receptors) are TLRs, or a portion thereof, such as the part of TLRs external to the host cell (the extracellular domain, eTLR). Since the interactions of PAMPs with one or more TLRs are multimeric, the binding of PAMPs causes receptor aggregation and leads to fluorescence resonance energy transfer (FRET) of reporter dyes that are attached to the TLRs or eTLRs.

This membrane-based FRET assay approach using planar optical waveguides has been demonstrated and validated with cholera toxin (Kelly et al., Opt Lett 24:1723-1725, 1999). The natural glycolipid receptor for cholera toxin, GM1, was fluorescently labeled with either a FRET donor or acceptor fluorophore. Cholera can bind up to five GM1 receptors, therefore the cholera-GM1 binding results in receptor aggregation and fluorescence resonance energy transfer of the reporter dyes. Upon introduction of the toxin and resultant receptor aggregation, the fluorescence emission of the donor molecules decreases (peak at 575 nm), whereas the fluorescence intensity of the acceptor molecules increases (peak at 620 nm).

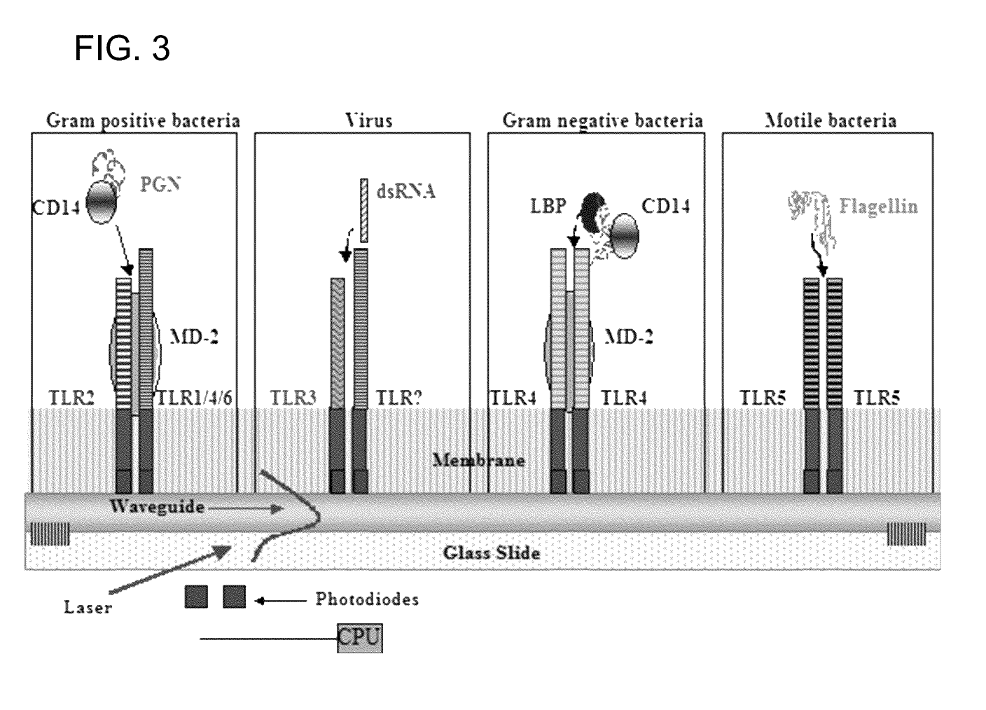

Having successfully validated membrane-based FRET for sensing, the present disclosure describes the next generation of biosensor, a sensor that detects the earliest signs of infection. In particular, this disclosure describes the development of a TLR-based sensor array for the early detection of infection (illustrated in FIG. 3). In addition to laying the foundation for a new detection modality for the early diagnosis of infection, the described TLR array provides a unique opportunity to address important and timely issues in the field of innate immunity. The ability to define and tailor the molecular make-up of a membrane bilayer allows one to determine the specificities of the individual TLRs and their combinations for the recognition of different PAMPs, thereby providing novel insights into the mechanisms of TLR-mediated recognition. Many as yet unknown PAMPs are involved in triggering the innate immune response. TLR arrays enable the discovery of new PAMP-TLR pairs.

This sensing platform can be adapted to many different pathogenic markers through the use of different types of man-made receptors (antibodies, peptides and carbohydrates). In order to incorporate these receptors into the membrane, a class of generic membrane anchors with differing hydrophilic spacers and dyes has been generated. The generic membrane anchor shown in FIG. 4 builds on a trifunctional amino acid core to attach three different moieties (receptor, reporter dye and membrane anchor). In some embodiments of the present disclosure, the extracellular domains of recombinant TLRs (eTLRs) are linked to these membrane anchoring molecules, and then incorporated into membranes to examine their PAMP-mediated aggregation.

The approach described herein mimics TLRs in nature without the molecular complexity present on the cell surface. The use of multiple receptors incorporated in a laterally fluid membrane provides a large increase in the effective TLR-PAMP binding affinities (Berzofsky et al., Fundamental Immunology, ed. Paul, W. E., pages 75-110, Lippincott-Raven, New York, 1998; Mammen et al., Angew Chem Int Edit 37:2755-2794, 1998). This results from the very high effective concentrations of proteins and receptors on a surface that enhance multiple binding reactions (Kelly et al., Opt Lett 24:1723-1725, 1999).

As described below, modified eTLRs are generated and conjugated to the generic membrane anchor, which are then incorporated into bilayers. The eTLR bilayers are used to study the molecular scaffolds that are formed upon binding purified PAMPs and assays are optimized for the detection of specific PAMPs. In parallel, eTLR-PAMP interactions are studied using microwell arrays and microscopic interrogation, as well as optical waveguide array approaches that form the basis for a compact sensor system. These array based approaches can be used for real-world samples, such as for clinical diagnostics.

A. Production of eTLR Proteins and Bioconjugation

TLRs are single transmembrane proteins containing an extracellular domain, a short membrane spanning region and a cytosolic (intracellular) portion. The presence of PAMPs is detected by the extracellular domain, which aggregates upon binding. This aggregation signals the presence of PAMPs to the cytosolic domain initiating a series of intracellular signals. Cell surface receptors most often have a series of post-translational modifications such as glycosylations, which play an important role in recognition by extracellular ligands. The importance of such modifications in PAMP-recognition has recently been shown for TLR4 (Correia and Ulevitch, J Biol Chem 277:1845-1854, 2002). For the studies described herein, the extracellular domains of human TLRs are recombinantly produced using mammalian or insect (baculoviral) (Iwaki et al., J Biol Chem 277:24315-24320, 2002) expression systems, both of which allow for correct folding and post-translational modifications of large membrane associated mammalian proteins.

Standard molecular biology techniques and commercially available reagents are used to produce eTLR proteins that in some embodiments are modified with a signal peptide at the amino-terminus to promote protein secretion from the cell to the cell culture medium. In some cases, a dicysteine moiety is added at the carboxy-terminus to direct conjugation to the membrane anchor, and a commonly used small amino acid-based affinity tag is added to aid in purification of the protein from the complex culture medium. In brief, mammalian or insect cells are transiently transfected with nucleic acid that encode the eTLR proteins containing the above described modifications. The particular procedures are different for expression in mammalian and insect cells, but both are standard methods. The transfected cells secrete the eTLRs into the growth medium that will contain other proteins. The growth medium is tested for the presence of the tagged eTLRs using commercial antibodies against the affinity-tag and TLRs. Non-transfected cells serve as a control. Because the eTLRs of interest are affinity-tagged, they can be purified from the protein mixture using affinity columns. The eluted fractions are examined for the presence of eTLRs and for purity as described above. Fractions containing the pure eTLRs are pooled, dialyzed into a suitable buffer and their protein concentration is determined.

eTLRs are conjugated to the membrane anchoring molecules described above for incorporation into the membrane-based FRET assay. It has previously been determined that bioconjugation of antibodies and other proteins to the membrane anchor is difficult to achieve in aqueous media owing to the hydrophobic nature of the membrane anchor and its poor solubility in water. If, however, the membrane anchor is first incorporated into vesicles and then exposed to proteins, bioconjugation is efficient. Fluorescence correlation spectroscopy (FCS) can be used to track and optimize this bioconjugation step for the efficient use of antibodies. The same approach is used in optimizing the bioconjugation of eTLRs to the membrane anchoring molecules. The membranes with eTLR receptors on glass and waveguide surfaces are characterized for uniformity and mobility with microscopic imaging techniques and fluorescence recovery after photobleaching (FRAP) (Axelrod et al., Biophys J 16:1055-1069, 1976).

B. Studies of Molecular Scaffolds

The binding of PAMPs to individual eTLRs and combinations of eTLRs can be explored to understand their homo- and heterodimerization upon recognition. The binding affinities/avidities of commercial purified PAMPs to fluorescently labeled, membrane-anchored eTLRs in vesicles or on supported bilayers can be determined. A set of complementary techniques, such as flow cytometry, surface plasmon resonance (SPR), fluorimetry and FCS, can also be used. These methods combined can measure interactions over the entire spectrum of affinities/avidities relevant to biology and are excellently suited for kinetic studies.

Three distinct TLR-PAMP scaffolds can be studied. The first is homodimerization of one type of TLR. In this case, the optimal detection scheme is to use proximity based fluorescence quenching. TLR5 binding to flagellin can be used as a model system for this type of scaffold. The second is a homodimerization of one type of TLR that requires the presence of a co-receptor to form and stabilize the scaffold. In this case, the optimal signal is FRET as the TLR and the co-receptor can be labeled with differing dyes. An exemplary model system for this type of scaffold is TLR4 and CD14 recognition of LPS. The third is heterodimerization where labeling of the two distinct TLRs with donor and acceptor dyes can be used to generate a FRET signal. As one example, the interaction of purified flagellin with eTLR5 can be analyzed. Using fluorimetry, fluorescence quenching upon binding of flagellin to membranes that have labeled eTLR5 embedded can be monitored. Varying the ratio of eTLRs to POPC and titrations against increasing concentrations of flagellin is used to optimize the assay. These studies also provide estimates for the affinity (single site interaction) and/or avidity (multivalent interaction) of the system. These experiments can also be performed using FCS, or by flow cytometry using lipid bilayers adsorbed on glass beads.

The binding of other TLRs for which PAMPs have been identified can further be explored. Some of these systems require the presence of other host receptors that are either membrane-anchored or in the extracellular environment. One example is TLR4 that requires the presence of CD14 and MD-2. MD-2 is a soluble protein while CD14 exists both as a membrane-bound and as a soluble protein. Membrane-bound CD14 is a GPI-linked protein and can be inserted into the outer leaf of our bilayer. Alternately, CD14 can be expressed with a carboxy-terminal cysteine residue and can be conjugated to the membrane anchor that is labeled with either donor or acceptor dye. LPS-induced homodimerization of two TLR4's in the absence of CD14 can be monitored by fluorescence quenching. Aggregation of eTLR4's in the presence of one CD14 each labeled with a different dye is followed using FRET. The relative concentrations of eTLR4 and CD 14 and their concentrations relative to POPC are varied to optimize the assay.

For TLRs that bind PAMPs as heterodimers, one is labeled with a donor and the other is labeled with an acceptor, and binding is followed using FRET. The assays are optimized as described above. For the above FRET-based assays, the use of two dyes results in a background that arises because of direct excitation of the acceptor dye. An interesting approach to reduce the background is the two-tiered FRET that minimizes the direct excitation of the acceptor fluorophore and thereby increases the assay sensitivity (Song et al., Anal Biochem 291:133-141, 2001). These “two-tier” approaches use three fluorophores, with two recognition molecules and are optimized similar to the two fluorophore energy transfer assays.

C. Development of Test TLR Arrays

Two exemplary approaches for the detection of PAMPs using eTLR arrays are described herein. The first relies on traditional microscopic analysis using a microwell array fabricated from glass. In this case, excitation and detection of the fluorescence signal is done using a traditional confocal microscope looking through the sample solution. This approach can be used to investigate and down-select from the many different combinations of eTLR pairs. The second focuses on the development of waveguide arrays that can be integrated into compact detection systems. It is possible to rapidly perform optimization experiments in a highly parallel fashion by using planar arrays of lipid membranes displaying various TLR combinations. On a single test “chip” several concentrations of the various combinations of eTLRs can be tested against the same PAMP concentration. The massive parallelism provided in this approach is useful in the rapid reduction of biosensor optimization parameter space.

Using varying concentrations of all available pure PAMPs, the entire pattern of response of an eTLR array can be analyzed in order to gain a further level of differentiation or classification. In particular, canonical discriminant analysis or PCA (principle component analysis) can be used from the multi-dimensional data sets generated by the array tests to help classify and distinguish pathogens. Canned clustering/classification algorithms can be used for further refinement. This analysis provides a pattern of expected responses for pure PAMPs and their levels of detection. In addition to pure PAMPs, such as host cell proteins (e.g. Hsp70 and other cell necrosis markers), responses from whole microorganisms can be tested.

D. Waveguide Based Sensor Arrays

To address real-world needs in medical surveillance, detection approaches that are amenable to integration within compact, easy-to-use, and inexpensive sensor systems are desirable. The detection approach described herein, evanescent excitation and fluorescence signal outcoupling using planar optical waveguides, permits direct analysis of medical samples, and simultaneous detection of multiple PAMPs. A compact, single channel optical biosensor has recently been developed by the inventors. The array size will be driven by the number of channels required for robust PAMP pattern recognition and physical constraints imposed by the waveguide chip. To permit patterning of membranes with different types and concentrations of eTLR receptors onto a single sensor substrate, micro-fabricated fluidic systems can be developed. Sensor arrays based on planar optical waveguides offer an attractive platform for membrane-based sensors. Advantages include: (1) spatial filtering of background fluorescence present in biological samples, (2) elimination of background from solution Raman scattering, (3) ease of membrane deposition and patterning to form multiple channels on a single substrate and (4) ability to provide small, inexpensive, modular sensor elements that can be easily interchanged. Planar optical waveguides are composed of a high index dielectric film deposited on a planar substrate. The evanescent field (portion of light that extends beyond the waveguide surface) provides optical excitation of reporter dyes within the membrane on the waveguide surface. A single mode waveguide supports several thousand reflections per cm of beam propagation providing a very intense evanescent field at the waveguide surface. The evanescent field intensity falls off exponentially away from the surface with no appreciable light beyond ˜200-300 nm. As a result, very little light penetrates into the sample region, minimizing background auto-fluorescence, yet the intensity at the membrane is very high. This high field strength is important when exciting a relatively low concentration of reporter dyes.

Single mode, high index waveguides can also provide for efficient backcoupling of the fluorescent signal from membrane into the waveguide. Backcoupling into a channel waveguide can provide integration of the signal along the path length and allow easy routing of the signal to detector(s). Several investigators have modeled the coupling of light emitted from surface dipoles back into the waveguide (Marcuse, Lightwave Technology 6:1273-1279, 1988; Fortune and Hall, J Opt Soc Am B-Optical Physics 19:860-869, 2002). Radiating dipoles in the sample solution are not coupled back into the waveguide providing even further discrimination from sample auto-fluorescence. The larger the index mismatch between the waveguide and the sample the greater the fluorescence backcoupling into the waveguide (Fortune and Hall, J Opt Soc Am B-Optical Physics 19:860-869, 2002; Chance, R. R. Molecular Fluorescence and Energy Transfer near Interfaces, in Advances in Chemical Physics, pages 1-65, Whiley: NY, 1978) suggesting that the signal strength obtained by backcoupling will be much higher than microscopic detection. To achieve a highly reproducible and facile coupling of laser light into the waveguide, grating coupling is employed in the single channel instrument. Gratings will be used for incoupling of excitation light as well as for spectral dispersion and outcoupling of emission fluorescence to detector(s). This approach has fundamental differences to other planar waveguide sensors currently under investigation (Rowe, Anal Chem 71:3846-3852, 1999). Differences include the use of single mode, high index waveguides (v.s. low index, multimode waveguides) for evanescent field enhancement and emission signal backcoupling for decreased detection system complexity (vs. cooled CCD arrays used in other efforts).

For film deposition, a vapor deposition technique known as flow-through ion deposition (FTID) can be used (Springer et al., Fusion Technology 31:449-455, 1997). FTID allows one to deposit very dense films of exceptional smoothness (<1-2 nm RMS) with good optical quality to minimize light loss in propagation. The goal is to optimize and extend current waveguide modeling and fabrication capabilities to investigate high index material (including tantala, titania and alumina, n˜1.66-2.6), multiple channel waveguide structures that facilitate backcoupling of emission signals into the waveguide.

For patterning and deposition of membranes, the waveguide surface must be exposed to a vesicle solution. Once formed, the membrane cannot be exposed to air. Micro-fluidic systems enabling patterned deposition of different membranes onto a single substrate and sample delivery to the sensor array will be accomplished using microfluidic multifunctional cartridges. A majority of the fabrication will be achieved using direct laser writing technique (infrared CO₂ laser) in glass and polymer materials.

Detection systems based on commercially available, palm size optical spectrometer(s) can be used. Although the single channel device employs silicon photodiodes with band-pass filters for detection, the present methods uses spectrometer based detection coupled with signal processing techniques to increase system signal to noise ratios allowing one to detect small changes in the spectral output induced by eTLR-PAMP. A spectrometer based detection system also provides flexibility in the selection and modification of excitation wavelengths and reporter dyes used within the membrane.

E. Applications

In the physiological scenario, when the host encounters a pathogen, the PAMPs are surrounded by various pathogenic and host molecules (sugars, lipids, proteins), and are not presented “purified” to the host cell. Thus, the present biosensor effort is able to show PAMP recognition and distinction in this physiological scenario. Thus far, the research in PAMP recognition has been limited to the use of purified PAMPs, ignoring pathogen-specific fingerprints. Three distinct categories of pathogens will be investigated to demonstrate organism-dependent, distinct, yet recognizable patterns of response: influenza virus (enveloped ssRNA orthomyxovirus), Escherichia coli (a motile, gram-negative bacterium) and Staphylococcus aureus (a gram-positive bacterium). Based on prior knowledge of TLR-PAMP recognition, there are certain expectations of the recognition profile of these three pathogens on the array. During an influenza infection, double stranded RNA (dsRNA, PAMP for TLR3) is produced during viral replication while peptidoglycan (PG, PAMP for TLR2) is a major component of the bacterial cell wall of Staphylococcus aureus. The Escherichia coli cell wall contains the PAMPs, LPS (for TLR4) and flagellin (for TLR5). Thus, disruption of these three pathogens by different means (e.g., hypotonic lysis, mechanical, extraction) will generate PAMPs that show some recognizable and predictable signatures, along with some novel and unexpected profiles from unidentified PAMPs. The pathogen fingerprints is characterized for each of these three organisms, and this information is used for the identification of these pathogens from clinical samples.

The following example is provided to illustrate certain particular features and/or embodiments; it should not be construed to limit the disclosure to the particular features or embodiments described.

### EXAMPLES

### Example 1

### Insertion of eTLR4 into a Lipid Bilayer

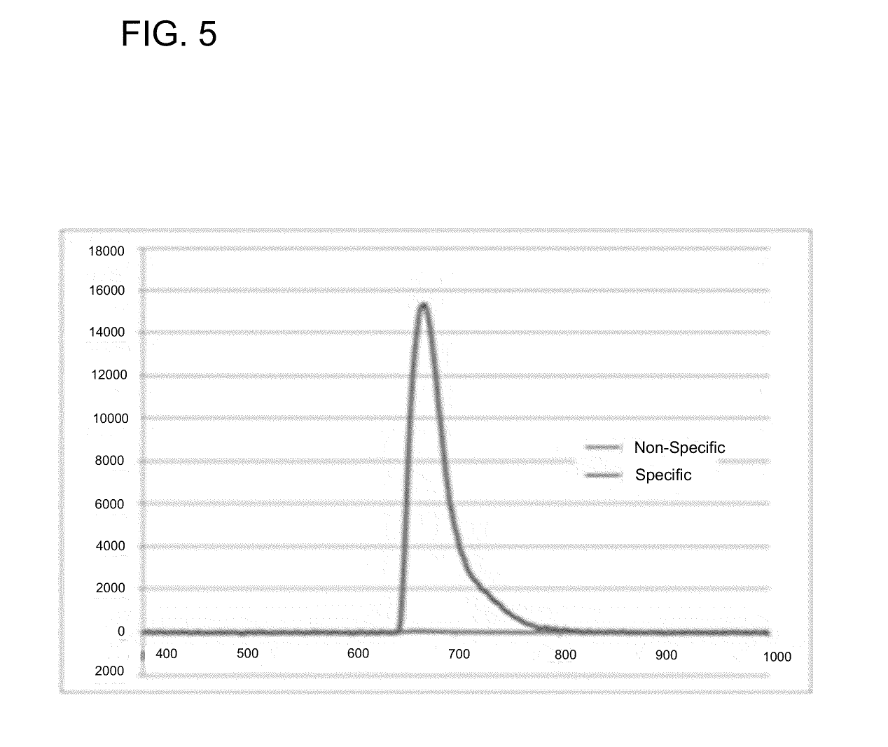

eTLR4 was purified in the laboratory with only a portion of its transmembrane region. The receptor was added (flooded) through a waveguide with a supported DOPC bilayer containing 0.1% biotin in it. A fluorescently labeled eTLR4 antibody was then added. Fluorescence indicated that the receptor was definitely associated (partitioned/inserted) into the surface (FIG. 5).

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method of detecting the presence of at least one pathogen-associated molecular pattern (PAMP) molecule or putative PAMP molecule in a biological sample, comprising:
providing a lipid assembly, wherein the lipid assembly comprises one or more toll-like receptors (TLRs) partitioned into or associated with the lipid assembly;
exposing the lipid assembly to the biological sample for an amount of time sufficient for the at least one PAMP molecule or putative PAMP molecule to bind the TLRs; and
detecting binding of the PAMP or putative PAMP to the TLRs, thereby detecting the presence of the PAMP or putative PAMP in the biological sample.

2. A method of diagnosing a subject as having a microbial infection, comprising detecting the presence of at least one PAMP molecule in a biological sample from the subject according to the method of claim 1, wherein the presence of the PAMP molecule in the biological sample diagnoses the subject as having a microbial infection.

3. The method of claim 2, wherein the microbial infection is a bacterial infection.

4. The method of claim 3, wherein the bacteria are Gram-positive.

5. The method of claim 3, wherein the bacteria are Gram-negative.

6. The method of claim 3, wherein the bacteria are motile bacteria.

7. The method of claim 3, wherein the microbial infection is a viral infection.

8. A method of capturing at least one PAMP molecule or putative PAMP molecule from a biological sample, comprising:
providing a lipid assembly, wherein the lipid assembly comprises one or more TLRs partitioned into or associated with the lipid assembly;
exposing the lipid assembly to the sample for an amount of time sufficient for the at least one PAMP molecule or putative PAMP molecule to bind the TLRs;
harvesting the lipid assembly with the one or more bound PAMP molecules or putative PAMP molecules; and
separating the one or more PAMP molecules or putative PAMP molecules from the lipid assembly.

9. A method of assessing disease state in a first subject, comprising
providing a biological sample from the first subject;
analyzing the biological sample using the method of claim 8 to produce a test PAMP profile for the sample;
comparing the test PAMP profile for the sample with a second PAMP profile, which PAMP profile is for a second sample selected from a sample taken from the first subject at a different time point; and a sample taken from a second subject; and
drawing a conclusion about the disease state of the first subject based on differences or similarities between the test PAMP profile and the second PAMP profile.

10. The method of claim 1, wherein the lipid assembly comprises a substantially planar lipid structure, a vesicle, a liposome, a nanodisc, a bicelle, or a micelle.

11. The method of claim 10, wherein the substantially planar lipid structure comprises a supported lipid bilayer (SLB), a tethered bilayer lipid membrane (t-BLM), a self-assembled monolayer (SAM), or a combination thereof.

12. The method of claim 11, wherein the substantially planar lipid structure is upon a functionalized waveguide surface.

13. The method of claim 10, wherein the vesicle is a multilamellar vesicle, a unilamellar vesicle, or a mixture thereof.

14. The method of claim 1, wherein the PAMP molecule or putative PAMP molecule is selected from the group consisting of cardiolipin, culture filtrate protein 10 (CFP-10), di-acyl lipopeptide, flagellin, lipoteichoic acid, lipid A, lipoarabinomannan (LAM), lipomannan, lipopolysaccharide (LPS), mycobactin T, peptidoglycan, phenolic glycolipid I (PGL-I), and tri-acyl lipopeptide.

15. The method of claim 1, wherein the putative PAMP molecule is characterized by stimulating TLR pathway activity in a cell-based TLR activity assay, being present in a sample from a subject exposed to or infected by a bacterial pathogen, or both.

16. The method of claim 1, wherein the biological sample is from a subject suspected of being infected with an infectious microorganism.

17. The method of claim 1, wherein the biological sample is a biological fluid sample.

18. The method of claim 17, wherein the biological fluid sample is a blood or blood product sample.

19. The method of claim 1, wherein the biological sample is a cell or tissue sample.

20. The method of claim 1, wherein the one or more TLRs are selected from TLR1, TLR2, TLR3, TLR4, TLR5, TLR6, TLR7, TLR8, TLR9, TLR10, TLR11, TLR12 and TRL13.

21. The method of claim 20, wherein the one or more TLRs comprise TLR4.

22. The method of claim 1, wherein the one or more TLRs are recombinant TLRs comprising or consisting of the extracellular domain of the TLRs (eTLRs).

23. The method of claim 1, wherein the one or more TLRs are conjugated to a membrane anchor which is inserted into the lipid assembly.

24. A synthetic lipid assembly comprising a substantially planar lipid structure, wherein the lipid assembly comprises one or more TLRs partitioned into or associated with the lipid assembly.

25. The synthetic lipid assembly of claim 25, wherein the substantially planar lipid structure comprises a supported lipid bilayer (SLB), a tethered bilayer lipid membrane (t-BLM), a self-assembled monolayer (SAM), or any combination thereof.

26. A biosensor comprising a lipid assembly on a functionalized waveguide surface, wherein the lipid assembly comprises one or more TLRs partitioned into or associated with the lipid assembly.

27. The biosensor of claim 26, wherein the lipid assembly comprises a substantially planar lipid structure, a vesicle, a liposome, a nanodisc, a bicelle, or a micelle.

28. The biosensor of claim 26, wherein the substantially planar lipid structure comprises a SLB, a t-BLM, a SAM, or any combination thereof.
